(19) United States
(12) Patent Application Publication
(10) Pub. No.: US 2018/0134657 A1
(43) Pub. Date: May 17, 2018

(54) BISMALEIMIDE RESINS FOR ONE DROP FILL SEALANT APPLICATION

(71) Applicant: Henkel IP & Holging GmbH, Duesseldorf (DE)

(72) Inventors: Laxmisha M. Sridhar, Monmouth Junction, NJ (US); John G. Woods, Farmington, CT (US)

(21) Appl. No.: 15/871,009

(22) Filed: Jan. 14, 2018

Related U.S. Application Data
(63) Continuation of application No. PCT/US2016/040611, filed on Jul. 1, 2016.
(60) Provisional application No. 62/192,086, filed on Jul. 14, 2015.

(51) Int. Cl. C07D 207/404; C08G 65/38; C09J 171/12; C07D 209/48; C08G 73/12; C09J 179/04

(52) U.S. Cl. CPC C07D 207/404; C08G 65/38; C09J 171/12; G02F 1/1339; C08G 73/124; C09J 179/04; C07D 209/48

(57) ABSTRACT

The present invention relates to curable novel bismaleimide resins and prepolymers, methods of manufacture. Particularly useful applications include one drop fill sealant used in liquid crystal assembly. In particular, the inventive polymers and compositions are useful in the assembly of LCD panels.

### BACKGROUND

### Field

The present invention relates to monomers and oligomers useful as sealants and particularly as one drop fill sealants for liquid crystal applications. In particular, the present invention permits assembly of LCD panels without migration of the sealant resin into the liquid crystal or vice versa during LCD assembly and/or curing of the resin.

### Brief Description of Related Technology

The one drop fill (“ODF”) process is becoming the mainstream process in the assembly of LCD panels in display applications, replacing the conventional vacuum injection technology to meet faster manufacturing process demands. In the ODF process, first, a sealant is dispensed on an electrode-equipped substrate to form a frame of a display element, and liquid crystals are dropped inside the depicted frame. In the next step of the assembly, another electrode equipped substrate is joined thereto under vacuum. Then, the sealant undergoes a curing process, either by a combination of UV and thermal or by thermal only process.

The ODF method has a few problems in that the sealant material in the uncured state comes into contact with the liquid crystal during the assembly process. This could cause reduction in electro-optical properties of the liquid crystal by resin migration into the liquid crystal or vice versa, or because of ionic impurities that may be present. Hence, design of resin systems for sealant material that show good liquid crystal resistance (less contamination) along with good adhesion and moisture barrier properties has remained a challenge.

### SUMMARY

The present invention relates to unique resins and ODF compositions made therefrom.

In one aspect of the invention there is provided a resin comprising the structure I:

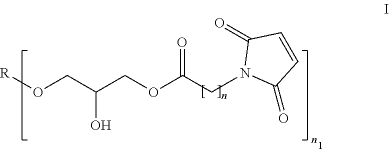

### Wherein:

R is a multivalent hydrocarbyl linker selected from linear or branched alkyls, linear or branched cycloalkyls, alkylenes, cycloalkylenes, bicycloalkylenes, tricycloalkylenes, linear or branched alkylenes, linear or branched cycloalkylenes, linear or branched alkenylenes, arylenes, aralkylenes, arylbicycloalkylenes, aryltricycloalkylenes, bicycloalkylarylenes, tricycloalkylarylenes, bisphenylenes, cycloalkylarylenes, heterocycloalkylene or heterocycloarylenes; the alkyls, cycloalkyls, alkylenes, cycloalkylenes, alkenylenes, arylenes, aralkylenes, arylbicycloalkylenes, aryltricycloalkylenes, bicycloalkylarylenes, tricycloalkylarylenes, bisphenylenes, cycloalkylarylenes, heterocycloalkylene and heterocycloarylenes can optionally contain O or S or hydroxyl group;
n and n₁ are each independently 1-10.

In another aspect of the invention there is included a resin having the structure II:

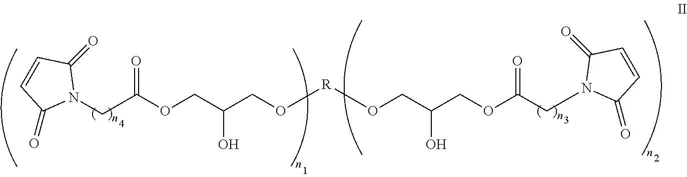

### Wherein:

R is a multivalent hydrocarbyl linker selected from linear or branched alkyls, linear or branched cycloalkyls, alkylenes, cycloalkylenes, bicycloalkylenes, tricycloalkylenes, linear or branched alkylenes, linear or branched cycloalkylenes, linear or branched alkenylenes, arylenes, aralkylenes, arylbicycloalkylenes, aryltricycloalkylenes, bicycloalkylarylenes, tricycloalkylarylenes, bisphenylenes, cycloalkylarylenes, heterocycloalkylene or heterocycloarylenes; the alkyls, cycloalkyls, alkylenes, cycloalkylenes, alkenylenes, arylenes, aralkylenes, arylbicycloalkylenes, aryltricycloalkylenes, bicycloalkylarylenes, tricycloalkylarylenes, bisphenylenes, cycloalkylarylenes, heterocycloalkylene and heterocycloarylenes can optionally contain O or S or hydroxyl group; and
n₁, n₂, n₃, and n₄ are each independently 1-10.

In another aspect of the invention there is included a resin having the structure III:

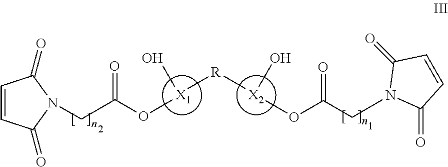

### Wherein:

X₁ and X₂ are each 3-10 membered rings independently selected from functionalized or unfunctionalized alicycyclic groups optionally having one or more heteroatoms;
n₁ and n₂ are each independently 1-10;
R is a multivalent hydrocarbyl linker selected from linear or branched alkyls, linear or branched cycloalkyls, alkylenes, cycloalkylenes, bicycloalkylenes, tricycloalkylenes, linear or branched alkylenes, linear or branched cycloalkylenes, linear or branched alkenylenes, arylenes, aralkylenes, arylbicycloalkylenes, aryltricycloalkylenes, bicycloalkylarylenes, tricycloalkylarylenes, bisphenylenes, cycloalkylarylenes, heterocycloalkylene or heterocycloarylenes; the alkyls, cycloalkyls, alkylenes, cycloalkylenes, alkenylenes, arylenes, aralkylenes, arylbicycloalkylenes, aryltricycloalkylenes, bicycloalkylarylenes, tricycloalkylarylenes, bisphenylenes, cycloalkylarylenes, heterocycloalkylene and heterocycloarylenes can optionally contain O or S or hydroxyl group; and
R is linked to the ring structures X₁ and X₂ at any position with a proviso that the hydroxyl groups on X₁ and X₂ rings are adjacent to the maleimidoalkanoyl groups.

In another aspect of the invention there is included a resin having the structure IV:

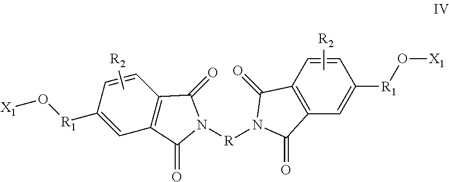

### Wherein:

R is a multivalent hydrocarbyl linker selected from linear or branched alkyls, linear or branched cycloalkyls, alkylenes, cycloalkylenes, bicycloalkylenes, tricycloalkylenes, linear or branched alkylenes, linear or branched cycloalkylenes, linear or branched alkenylenes, arylenes, aralkylenes, arylbicycloalkylenes, aryltricycloalkylenes, bicycloalkylarylenes, tricycloalkylarylenes, bisphenylenes, cycloalkylarylenes, heterocycloalkylene or heterocycloarylenes; the alkyls, cycloalkyls, alkylenes, cycloalkylenes, alkenylenes, arylenes, aralkylenes, arylbicycloalkylenes, aryltricycloalkylenes, bicycloalkylarylenes, tricycloalkylarylenes, bisphenylenes, cycloalkylarylenes, heterocycloalkylene and heterocycloarylenes can optionally contain O or S or hydroxyl group;
R₁ can be a carbonyl; an aliphatic or aromatic linker and may contain one or more of ester, ether, hydroxyl or thioether groups;
R₂ is a substituent on the aromatic ring, which can be H, halogen, alkyl, alkyl ether, thioether group; and
X₁ is selected from maleimidoalkanoyl or maleimidoaroyl group.

In another aspect of the invention there is included a resin having the structure V:

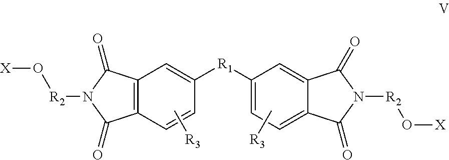

### Wherein:

R₁ can be just a bond linking the two aromatic groups; O; carbonyl; or a multivalent hydrocarbyl linker selected from linear or branched alkyls, linear or branched cycloalkyls, alkylenes, cycloalkylenes, bicycloalkylenes, tricycloalkylenes, linear or branched alkylenes, linear or branched cycloalkylenes, linear or branched alkenylenes, arylenes, aralkylenes, arylbicycloalkylenes, aryltricycloalkylenes, bicycloalkylarylenes, tricycloalkylarylenes, bisphenylenes, cycloalkylarylenes, heterocycloalkylene or heterocycloarylenes; the alkyls, cycloalkyls, alkylenes, cycloalkylenes, alkenylenes, arylenes, aralkylenes, arylbicycloalkylenes, aryltricycloalkylenes, bicycloalkylarylenes, tricycloalkylarylenes, bisphenylenes, cycloalkylarylenes, heterocycloalkylene and heterocycloarylenes can optionally contain O or S or hydroxyl group;
R₂ is an aliphatic or aromatic linker group which may contain one or more of ester, ether, hydroxyl, thioether or carbonate groups;
R₃ is a substituent on the aryl group, which may be H, halogen, alkyl, alkyl ether, or thio ether group; and
X is a polymerizable functionality selected from maleimidoalkanoyl and maleimidoaroyl groups.

In another aspect of the invention there is included a resin having the structure VI:

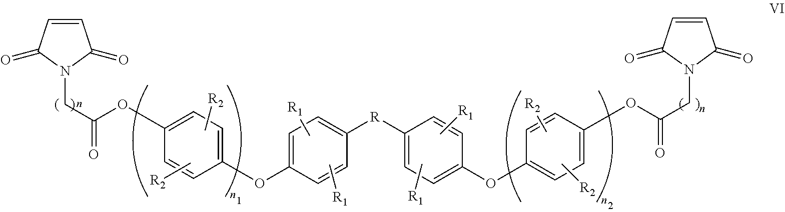

### Wherein:

R is a divalent hydrocarbyl linker selected from linear or branched alkyls, linear or branched cycloalkyls, alkylenes, cycloalkylenes, bicycloalkylenes, tricycloalkylenes, linear or branched alkylenes, linear or branched cycloalkylenes, linear or branched alkenylenes, arylenes, aralkylenes, arylbicycloalkylenes, aryltricycloalkylenes, bicycloalkylarylenes, tricycloalkylarylenes, bisphenylenes, cycloalkylarylenes, heterocycloalkylene or heterocycloarylenes; the alkyls, cycloalkyls, alkylenes, cycloalkylenes, alkenylenes, arylenes, aralkylenes, arylbicycloalkylenes, aryltricycloalkylenes, bicycloalkylarylenes, tricycloalkylarylenes, bisphenylenes, cycloalkylarylenes, heterocycloalkylene and heterocycloarylenes can optionally contain O or S or hydroxyl group;
R₁ and R₂ are each linear or branched aliphatic groups optionally containing heteroatoms;
n is 1-10; and n₁ and n₂ are 1-100.

### DETAILED DESCRIPTION

The polymers of the present invention are useful in a wide variety of applications including sealing, adhesion and coating. One particularly desirable use is as an ODF sealant for assembling LCD panels.

The present invention includes a number of novel materials including resins, oligomers and polymers useful for preparing curable compositions which may be used for ODF sealants. The present invention also includes novel compositions made from the disclosed resins. For purposes of this invention, the term “resins” will include the aforementioned the novel materials, i.e. resins, oligomers and polymers.

One aspect of the invention includes a curing resin composition for use as an ODF sealant, which includes resins represented by the general structural formulae shown above.

The glycidyl ether/ester compounds useful in synthesizing some of the inventive resins described herein is not particularly limited, and examples of the compounds available in the market include: bisphenol A type epoxy resins such as Epikote 828EL and Epikote 1004 (all manufactured by Japan Epoxy Resin Co., Ltd.); bisphenol F type epoxy resins such as Epikote 806 and Epikote 4004 (all manufactured by Japan Epoxy Resin Co., Ltd.); bisphenol S type epoxy resins such as Epiclon EXA1514 (manufactured by Dainippon Ink and Chemicals Inc.) and SE 650 manufactured by Shin A T&C; 2,2′-diallyl bisphenol A type epoxy resins such as RE-81 ONM (manufactured by Nippon Kayaku Co., Ltd.); hydrogenated bisphenol type epoxy resins such as Epiclon EXA7015 (manufactured by Dainippon Ink and Chemicals Inc.); propyleneoxide-added bisphenol A type epoxy resins such as EP-4000S (manufactured by ADEKA Corporation); resorcinol type epoxy resins such as EX-201 (manufactured by Nagase ChemteX Corporation); biphenyl type epoxy resins such as Epikote YX-4000H (manufactured by Japan Epoxy Resin Co., Ltd.); sulfide type epoxy resins such as YSLV 50TE (manufactured by Tohto Kasei Co., Ltd.); ether type epoxy resins such as YSLV 80DE (manufactured by Tohto Kasei Co., Ltd.); dicyclopentadiene type epoxy resins such as EP-40885 and EP4088L (manufactured by ADEKA Corporation); naphthalene type epoxy resins such as SE-80, SE-90, manufactured by Shin A T&C; glycidyl amine type epoxy resins such as Epikote 630 (manufactured by Japan Epoxy Resin Co., Ltd.), Epiclon 430 (manufactured by Dainippon Ink and Chemicals Inc.) and TETRAD-X (manufactured by Mitsubishi Gas Chemical Company Inc.); alkylpolyol type epoxy resins such as ZX-1542 (manufactured by Tohto Kasei Co., Ltd.), Epiclon 726 (manufactured by Dainippon Ink and Chemicals Inc.), Epolight 8OMFA (manufactured by Kyoeisha Chemical Co., Ltd.) and Denacol EX-611 (manufactured by Nagase ChemteX Corporation); rubber modified type epoxy resins such as YR-450,YR-207 (all manufactured by Tohto Kasei Co., Ltd.) and Epolead PB (manufactured by Daicel Chemical Industries, Ltd.); glycidyl ester compounds such as Denacol EX-147 (manufactured by Nagase ChemteX Corporation); bisphenol A type episulfide resins such as Epikote YL-7000 (manufactured by Japan Epoxy Resin Co., Ltd.); and others such as YDC-1312, YSLV-BOXY, YSLV-90CR (all manufactured by Tohto Kasei Co., Ltd.), XAC4151 (manufactured by Asahi Kasei Corporation), Epikote 1031, Epikote 1032 (all manufactured by Japan Epoxy Resin Co., Ltd.), EXA-7120 (manufactured by Dainippon Ink and Chemicals Inc.), TEPIC (manufactured by Nissan Chemical Industries, Ltd.). Examples of the commercially available phenol novolak type epoxy compound include Epiclon N-740, N-770, N-775 (all manufactured by Dainippon Ink and Chemicals Inc.), Epikote 152, Epikote 154 (all manufactured by Japan Epoxy Resin Co., Ltd.), and the like. Examples of the commercially available cresol novolak type epoxy compound include Epiclon N-660, N-665, N-670, N-673, N-680, N-695, N-665-EXP and N-672-EXP (all manufactured by Dainippon Ink and Chemicals Inc.); an example of the commercially available biphenyl novolak type epoxy compound is NC-3000P (manufactured by Nippon Kayaku Co., Ltd.); examples of the commercially available trisphenol novolak type epoxy compound include EP1032S50 and EP1032H60 (all manufactured by Japan Epoxy Resin Co., Ltd.); examples of the commercially available dicyclopentadiene novolak type epoxy compound include XD-1000-L (manufactured by Nippon Kayaku Co., Ltd.) and HP-7200 (manufactured by Dainippon Ink and Chemicals Inc.); examples of the commercially available bisphenol A type epoxy compound include Epikote 828, Epikote 834, Epikote 1001, Epikote 1004 (all manufactured by Japan Epoxy Resin Co., Ltd.), Epiclon 850, Epiclon 860 and Epiclon 4055 (all manufactured by Dainippon Ink and Chemicals Inc.); examples of the commercially available bisphenol F type epoxy compound include Epikote 807 (manufactured by Japan Epoxy Resin Co., Ltd.) and Epiclon 830 (manufactured by Dainippon Ink and Chemicals Inc.); an example of the commercially available 2,2′-diallyl bisphenol A type epoxy compound is RE-810NM (manufactured by Nippon Kayaku Co., Ltd.); an example of the commercially available hydrogenated bisphenol type epoxy compound is ST-5080 (manufactured by Tohto Kasei Co., Ltd.); examples of the commercially available polyoxypropylene bisphenol A type epoxy compound include EP-4000 and EP-4005 (all manufactured by ADEKA Corporation); and the like. HP4032 and Epiclon EXA-4700 (all manufactured by Dainippon Ink and Chemicals Inc.); phenol novolak type epoxy resins such as Epiclon N-770 (manufactured by Dainippon Ink and Chemicals Inc.); orthocresol novolak type epoxy resins such as Epiclon N-670-EXP-S (manufactured by Dainippon Ink and Chemicals Inc.); dicyclopentadiene novolak type epoxy resins such as Epiclon HP7200 (manufactured by Dainippon Ink and Chemicals Inc.); biphenyl novolak type epoxy resins such as NC-3000P (manufactured by Nippon Kayaku Co., Ltd.); and naphthalene phenol novolak type epoxy resins such as ESN-165S (manufactured by Tohto Kasei Co., Ltd.).

Examples of the alicyclic epoxy compounds useful in synthesizing some of the inventive resins include, without limitation, polyglycidyl ethers of polyhydric alcohols having at least one alicyclic ring and cyclohexene oxide- or cyclopentene oxide containing compounds obtained by epoxidizing cyclohexene ring or cyclopentene ring-containing compounds. Specific examples include hydrogenated bisphenol A diglycidyl ether, 3,4-epoxycyclohexylmethyl 3,4-epoxycyclohexanecarboxylate, 3,4-epoxy-1-methyl cyclohexyl-3,4-epoxy-1-methylcyclohexanecarboxylate, 6-methyl-3,4-epoxycyclohexylmethyl-6-methyl-3,4-epoxy-cyclohexanecarboxylate, 3,4-epoxy-3-methylcyclohexylmethyl 3,4-epoxy-3-methylcyclohexanecarboxylate, 3,4-epoxy-5-methylcylcohexylmethyl-3,4-epoxy-5-methylcyclohexanecarboxylate, 2-(3,4-epoxycyclohexyl-5,5-spiro-3,4-epoxy)cyclohexane-metadioxane, bis(3,4-epoxycyclohexylmethyl)adipate, 3,4-epoxy-6-methylcyclohexyl carboxylate, methylenebis(3,4-epoxycyclohexane), dicyclopentadiene diepoxide, ethylenebis(3,4-epoxycyclohexanecarboxylate), dioctylepoxyhexahydrophthalate, and di-2-ethylhexyl epoxyhexahydrophthalate.

Some of these alicyclic epoxy resins are commercially available as: UVR-6100, UVR-6105, UVR-6110, UVR-6128, and UVR-6200 (products of Dow Corporation); CELLOXIDE 2021, CELLOXIDE 2021P, CELLOXIDE 2081, CELLOXIDE 2083, CELLOXIDE 2085, CELLOXIDE 2000, CELLOXIDE 3000, CYCLMER A200, CYCLMER M100, CYCLMER M101, EPOLEAD GT-301, EPOLEAD GT-302, EPOLEAD 401, EPOLEAD 403, ETHB, and EPOLEADHD 300 (products of Daicel Chemical Industries, Ltd.); KRM-2110, and KRM-2199 (products of ADEKA Corporation).

In addition to the curable polymers of the present invention, ODF sealant compositions may also include a free radical initiator (thermal or UV generated) and a curing agent. Curing of the ODF compositions may be by thermal or UV mechanisms or both. In embodiments where an epoxide ring is present, a latent epoxy curing agent may also be employed.

Useful thermal free radical initiators include, for example, organic peroxides and azo compounds that are known in the art. Examples include: azo free radical initiators such as AIBN (azodiisobutyronitrile), 2,2′-azobis(4-methoxy-2,4-dimethyl valeronitrile), 2,2′-azobis(2,4-dimethyl valeronitrile), dimethyl 2,2′-azobis(2-ethylpropionate), 2,2′-azobis(2-methylbutyronitrile), 1,11-azobis(cyclohexane-1-carbonitrile), 2,2′-azobis[N-(2-propenyl)-2-methylpropionamide]; dialkyl peroxide free radical initiators such as 1,1-di-(butylperoxy-3,3,5-trimethyl cyclohexane); alkyl perester free radical initiators such as TBPEH (t-butyl per-2-ethylhexanoate); diacyl peroxide free radical initiators such as benzoyl peroxide; peroxy dicarbonate radical initiators such as ethyl hexyl percarbonate; ketone peroxide initiators such as methyl ethyl ketone peroxide, bis(t-butyl peroxide) diisopropylbenzene, t-butylperbenzoate, t-butyl peroxy neodecanoate, and combinations thereof.

Further examples of organic peroxide free radical initiators include: dilauroyl peroxide, 2,2-di(4,4-di(tert-butylperoxy)cyclohexyl)propane, di(tert-butylperoxyisopropyl) benzene, di(4-tert-butylcyclohexyl) peroxydicarbonate, dicetyl peroxydicarbonate, dimyristyl peroxydicarbonate, 2,3-dimethyl-2,3-diphenylbutane, dicumyl peroxide, dibenzoyl peroxide, diisopropyl peroxydicarbonate, tert-butyl monoperoxymaleate, 2,5-dimethyl-2,5-di(tert-butylperoxy)hexane, tert-butylperoxy 2-ethylhexyl carbonate, tert-amyl peroxy-2-ethylhexanoate, tert-amyl peroxypivalate, tert-amylperoxy 2-ethylhexyl carbonate, 2,5-dimethyl-2,5-di(2-ethylhexanoylperoxy) hexane 2,5-dimethyl-2,5-di(tert-butylperoxy) hexpe-3, di(3-methoxybutyl)peroxydicarbonate, diisobutyryl peroxide, tert-butyl peroxy-2-ethylhexanoate (Trigonox 21 S), 1,1-di(tert-butylperoxy)cyclohexane, tert-butyl peroxyneodecanoate, tert-butyl peroxypivalate, tert-butyl peroxyneoheptanoate, tert-butyl peroxydiethylacetate, 1,1-di(tert-butylperoxy)-3,3,5-trimethylcyclohexane, 3,6,9-triethyl-3,6,9-trimethyl-1,4,7-triperoxonane, di(3,5,5-trimethylhexanoyl) peroxide, tert-butyl peroxy-3,5,5-trimethyl hexanoate, 1,1,3,3-tetramethylbutyl peroxy-2-ethylhexanoate, 1,1,3,3-tetramethylbutyl peroxyneodecanoate, tert-butyl peroxy-3,5,5-trimethyl hexanoate, cumyl peroxyneodecanoate, di-tert-butyl peroxide, tert-butylperoxy isopropyl carbonate, tert-butyl peroxybenzoate, di(2-ethylhexyl) peroxydicarbonate, tert-butyl peroxyacetate, isopropylcumyl hydroperoxide, and tert-butyl cumyl peroxide.

Ordinarily the thermal free radical initiator with higher decomposition rate is preferred, as this can generate free radicals more easily at common cure temperature (80-130° C.) and give faster cure speed, which can reduce the contact time between liquid resin and liquid crystal, and reduce the liquid crystal contamination. On the other hand, if the decomposition rate of initiator is too high, the viscosity stability at room temperature will be influenced and thereby reducing the work life of the sealant.

A convenient way of expressing the decomposition rate of an initiator at a specified temperature is in terms of its half-life i.e., the time required to decompose one-half of the peroxide originally present. To compare reactivity of different initiators, the temperature at which each initiator has a half-life (T½) of 10 hours is used. The most reactive (fastest) initiator would be the one with the lowest 10 h T½ temperature.

The thermal free radical initiator with 10 h T½ temperature of 30-80° C. is preferred, and the thermal free radical initiator with 10 h T½ temperature of 40-70° C. is more preferred.

To balance the reactivity and viscosity stability of the composition, the thermal free radical initiator used in the resin composition is in an amount of usually 0.01 to 3 parts by weight, and preferably 0.5 to 2 parts by weight, based on 100 parts by weight of the inventive resin in the curable composition of the present invention.

Useful UV free radical initiators include Norrish type I cleavage photoinitiators that are commercially available from CIBA and BASF. These photoinitiators are used in the amount 0.1-5 wt %, more preferably in about 0.2 to 3 wt % in the formulation.

Examples of useful epoxy curing agent include but are not limited to the Ajicure series of hardeners available from Ajinomoto Fine-Techno Co., Inc.; the Amicure series of curing agents available from Air products and the JERCURE™ products available from Mitsubushi Chemical. These curing agents or hardeners or hardeners are used in the amount of about 1% to about 50% by weight of the total composition, more preferably from about 5% to about 20% by weight of the total composition.

The curable composition may optionally contain, as desired, a further component capable of a photopolymerization reaction such as a vinyl ether compound. In addition, the curable composition may further comprise additives, resin components and the like to improve or modify properties such as flowability, dispensing or printing property, storage property, curing property and physical property after curing.

Various additives may be contained in the composition as desired, for example, organic or inorganic fillers, thixotropic agents, silane coupling agents, diluents, modifiers, coloring agents such as pigments and dyes, surfactants, preservatives, stabilizers, plasticizers, lubricants, defoamers, leveling agents and the like; however it is not limited to these. In particular, the composition preferably comprises an additive selected from the group consisting of organic or inorganic filler, a thixotropic agent, and a silane coupling agent. These additives may be present in amounts of about 0.1% to about 50% by weight of the total composition, more preferably from about 2% to about 10% by weight of the total composition.

The filler may include, but is not limited to, inorganic fillers such as silica, diatomaceous earth, alumina, zinc oxide, iron oxide, magnesium oxide, tin oxide, titanium oxide, magnesium hydroxide, aluminium hydroxide, magnesium carbonate, barium sulphate, gypsum, calcium silicate, talc, glass bead, sericite activated white earth, bentonite, aluminum nitride, silicon nitride, and the like; meanwhile, organic fillers such as poly(methyl) methacrylate, poly(ethyl) methacrylate, poly(propyl) methacrylate, poly(butyl) methacrylate, butylacrylate-methacrylic acid-(methyl) methacrylate copolymer, polyacrylonitrile, polystyrene, polybutadiene, polypentadiene, polyisoprene, polyisopropylene, and the like. These may be used alone or in combination. These fillers may be present in amounts of about 1% to about 80%, more preferably from about 5% to about 30% by weight of the total composition.

The thixotropic agent may include, but is not limited to, talc, fume silica, superfine surface-treated calcium carbonate, fine particle alumina, plate-like alumina; layered compounds such as montmorillonite, spicular compounds such as aluminium borate whisker, and the like. Among them, talc, fume silica and fine alumina are particularly desired. These agents may be present in amounts of about 1% to about-50%, more preferably from about 1% to about 30% by weight of the total composition.

The silane coupling agent may include, but is not limited to, γ-minopropyltriethoxysilane, γ-mercaptopropyltrimethoxysilane, γ-methacryloxypropyltrimethoxysilane, γ-glycidoxypropyltrimethoxylsilane, and the like.

The curable composition according to the present invention may be obtained by mixing the aforementioned each component by means of, for example, a mixer such as a stirrer having stirring blades and a three roll mill. The composition is liquid at ambient with the viscosity of 200-400 Pa·s (at 25° C.) at 1.5 s-1 shear rate, which allows for easy dispensing.

Also provided is a method for manufacturing a liquid crystal display having a liquid crystal layer between a first substrate and a second substrate, by means of a liquid crystal one-drop-filling process. The method comprises the steps of
(a) applying the curable composition described in the present invention on a sealing region at periphery of a surface of the first substrate;
(b) dropping liquid crystal on a central area encircled by the sealing region of the surface of the first substrate;
(c) overlaying the second substrate on the first substrate;
(d) optionally performing partial curing by UV-irradiating the curable composition, and
(e) performing final curing by heating the curable composition.

The first substrate and the second substrate used in the present invention are usually transparent glass substrates. Generally, transparent electrodes, active matrix elements (such as TFT), alignment film(s), a color filter and the like are formed on at least one of the opposed faces of the two substrates. These constitutions may be modified according to the type of the LCD. The manufacturing method according to the present invention may be thought to be applied for any type of the LCD.

In step (a), the curable composition is applied on the periphery portion of the surface of the first substrate so as to lap around the substrate circumference in a frame shape. The portion where the curable composition is applied in a frame shape is referred as a seal region. The curable composition can be applied by a known method such as screen printing and dispensing.

In step (b), the liquid crystal is then dropped onto the center region surrounded by the seal region in the frame shape on the surface of the first substrate. This step is preferably conducted under reduced pressure.

In step (c), said second substrate is then placed over said first substrate, and UV-irradiated in the step (d). By the UV-irradiation, the curable composition cures partially and shows the strength at a level that displacement does not occur by handling, whereby the two substrates are temporally fixed. Generally, the radiation time is preferably short, for example not longer than 5 minutes, preferably not longer than 3 minutes, more preferably not longer than 1 minute.

In step (e), heating the curable composition allows it to achieve the final curing strength, whereby the two substrates are finally bonded. The thermal curing in the step (e) is generally heated at a temperature of 80 to 130° C., and preferably of 100 to 120° C., with the heating time of 30 mins to 3 hours, typically 1 hour.

By this process, the major part of the LCD panel is completed.

### Syntheses

General Procedure for Glycidyl Ether Ring Opening with 6-Maleimidocaproic Acid

In a round bottom flask equipped with a mechanical stirrer and nitrogen inlet were taken epoxy resin and appropriate stoichiometry of 6-maleimidocaproic acid in toluene. Methylhydroquinone (1000-3000 ppm) and Hycat 2000S epoxy ring opening catalyst (1 wt %) were added and the mixture stirred at 60° C. for about 24 h. After cooling to room temperature (room temperature), an appropriate amount of ethyl acetate was added and the mixture was washed twice with aqueous NaHCO₃ solution and several times with deionized water. After drying over anhydrous Na₂SO₄, the solvent was passed through a silica column. Another 500 ppm of methylhydroquinone was added and the solvent evaporated to give give inventive bismaleimides resins.

### Inventive Resin Syntheses

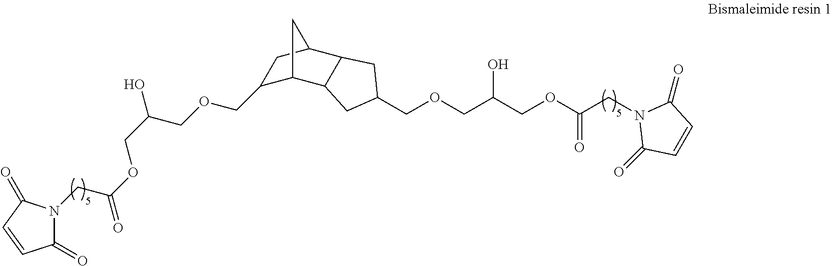

### Preparation of Inventive Bismaleimide Resin 1

In a 500 mL 4 necked flask equipped with a mechanical stirrer were taken EP 40885 (52.7 g, 171 mmol), 6-maleimidocaproic acid (75.8 g, 359 mmol), methylhydroquinone (60 mg, 500 ppm) in toluene (200 mL). The mixture was heated at 60° C. until it became homogenous. Hycat 2000S (1.28 g, 1 wt %) was added and the mixture stirred at the same temperature overnight (about 14 h). After cooling to r.t., 300 mL of ethyl acetate was added and the organic layer washed twice with aq. NaHCO₃ solution and several times with deionized water. After drying over anhydrous Na₂SO₄, the organic layer was passed through a column of silica gel and the solvent evaporated to give bismaleimide resin 1 (104 g, 81%).

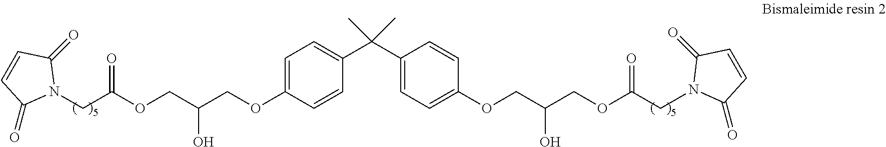

### Preparation of Inventive Bismaleimide Resin 2

In a 1 L 4 necked flask equipped with a mechanical stirrer were taken bisphenol A diglycidyl ether (122 g, 358 mmol), 6-maleimidocaproic acid (159 g, 752 mmol) in toluene (200 mL). The mixture was stirred at 60° C. until it became homogenous. Hycat 2000S (2.81 g, 1 wt %) was added and the mixture stirred at the same temperature overnight. 500 mL of ethyl acetate was added and the mixture decanted to a separatory funnel and washed twice with saturated aq. NaHCO₃ solution (200 ml×2) and several times with deionized water. After drying the organic layer over anhydrous Na₂SO₄, the organic layer was passed through silica gel and the solvent evaporated to give Bisphenol A based bismaleimide resin 2 (220 g, 79%).

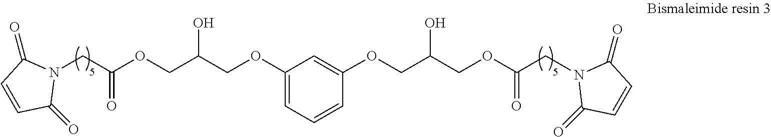

### Preparation of Inventive Bismaleimide Resin 3

In a 500 mL 4 necked flask equipped with a mechanical stirrer were taken resorcinol diglycidyl ether (RDGE) (66.5 g, 300 mmol), 6-maleimidocaproic acid (139 g, 658 mmol), methylhydroquinone (100 mg, 500 ppm) in toluene (200 mL) and the mixture was heated at 60° C. until it became homogenous. Hycat 2000S (2.05 g, 1 wt %) was added and the mixture stirred at 60° C. overnight. After cooling to r.t. 500 mL of ethyl acetate was added and the organic layer washed twice with saturated aq. NaHCO₃ solution (2×200 mL) and several times with deionized water. After drying over anhydrous Na₂SO₄, the organic layer was passed through a silica column to give bismaleimide resin 3 (175 g, 85%).

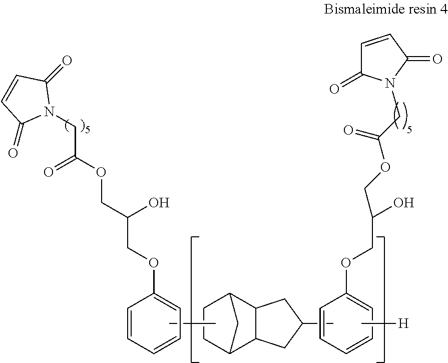

### Preparation of Inventive Bismaleimide Resin 4

In a 500 mL 4 necked flask equipped with a mechanical stirrer were taken Tactix 756 (92.5 g, 366 mmol, w.r.t epoxy functionality), 6-maleimidocaproic acid (80 g, 378 mmol), methylhydroquinone (87 mg, 500 ppm). Toluene (200 mL) was added and the mixture stirred at 60° C. until it became homogenous. Hycat 2000S was added (1.7 g, 1 wt %) and the mixture stirred at the same temperature for about 16 h. After cooling to r.t, 400 mL of ethyl acetate was added and the organic layer washed twice with aq. NaHCO₃ solution and several times with deionized water. After drying over anhydrous Na₂SO₄, the organic layer was passed through a silica column and the solvent evaporated to give the bismaleimide resin 4 (142 g, 82%).

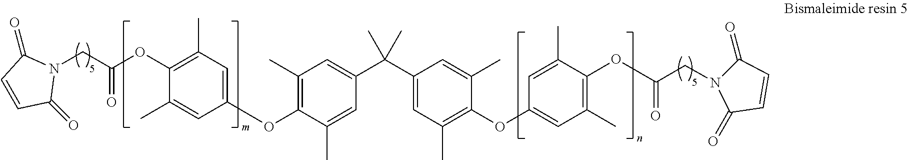

### Preparation of Inventive Bismaleimide Resin 5

In a 500 mL 4 necked flask equipped with a nitrogen inlet, mechanical stirrer was taken powdered 6-maleimidocaproic acid (26.2 g, 124.1 mmol). Trifluroacetic anhydride (26 g, 124 mmol) was added and the mixture stirred at r.t. for about 7 h. At this time the mixture became homogenous. Polyphenylene oxide PPO SA 90 (79.5 g, 49 mmol) was added followed by 60 mL of dichloromethane. The resulting mixture was stirred at r.t. overnight. A saturated aqueous NaHCO₃ solution was added and the mixture stirred for 30 min. 300 mL of ethyl acetate was added and the organic layer washed several times with deionized water and dried over anhydrous Na₂SO₄. The organic layer was passed through a column of silica gel to give the bismaleimide resin 5 as a brown solid (64 g, 61%).

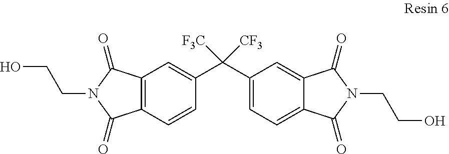 [SMILES: O=C1c2ccc(C(c3ccc4c(c3)C(=O)N(CCO)C4=O)(C(F)(F)F)C(F)(F)F)cc2C(=O)N1CCO]

### Preparation of Inventive Resin 6

4,4′-Hexaflurorisopropylidenediphthaleic anhydride (100 g, 225 mmol) was taken in a mixture of DMF (400 mL) and xylene (80 mL) in a 1 L 3 necked flask equipped with a mechanical stirrer and heating mantle. Ethanolamine (31 g, 506 mmol) was added at once (slightly exothermic, as the temp rose to about 45° C.). The mixture was heated to 170° C. as the reaction temperature gradually rose to about 139° C. when the azeotropic distillation started. The temperature eventually rose to about 160° C. in about 30 minutes. At this point, the reaction was stopped and IR indicated that the imidization reached completion. After cooling, 500 mL of water was added and stirred for 30 minutes. The precipitated solid was filtered off and washed several times with water and dried to give imide diol 6 as a light orange solid (101 g, 85%).

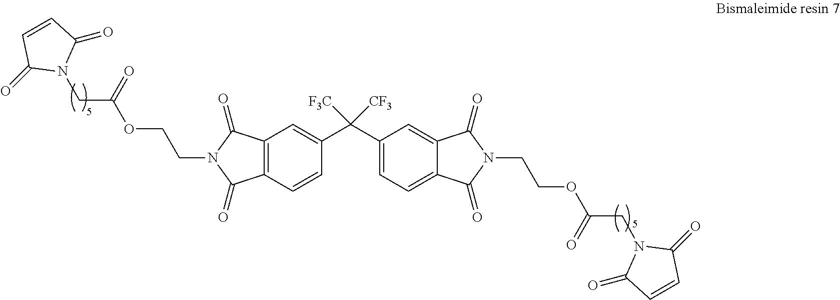

### Preparation of Inventive Bismaleimide Resin 7

In a 1 L 3 necked flask equipped with a mechanical stirrer and water condenser, were taken imide diol 6 (47.7 g, 89 mmol), 6-maleimidocaproic acid (45.6 g, 215 mmol), PTSA mono hydrate (1.71 g, 8.9 mmol), 4-methoxyphenol (100 mg, 1000 ppm) in toluene (400 mL). The mixture was refluxed with azeotrope distillation of water for about 7 h. After cooling to r.t., the mixture was diluted with ethyl acetate, washed with aq. NaHCO₃ solution twice, deionized water until the ionic conductivity was about 2 uS. The organic layer was passed through a silica column containing a short plug of sillitin in between the silica layers. Another 500 ppm pf 4-methoxyphenol was added and the solvent evaporated on rotovap to give bismaleimide resin 7 as a brown viscous liquid (72 g, 87%).

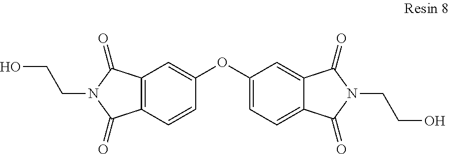 [SMILES: O=C1c2ccc(Oc3ccc4c(c3)C(=O)N(CCO)C4=O)cc2C(=O)N1CCO]

### Preparation of Inventive Resin 8

4,4′-Oxydiphthaleic anhydride (104 g, 335 mmol) was taken in a mixture of DMF (400 mL) and xylene (100 mL) in a 1 L 3 necked flask equipped with a mechanical stirrer and heating mantle. Ethanolamine (47 g, 769 mmol) was added at once (slightly exothermic, as the temp rose to about 48° C.). The mixture was heated to 170° C. as the reaction temperature gradually rose to about 139° C. when the azeotropic distillation started. The temperature eventually rose to about 170° C. in about 30 minutes. After most of the solvent has distilled off, the mixture was cooled to r.t. 500 mL of water was added and stirred well for 30 minutes. The precipitated white solid was filtered off, washed several times with water and dried to give the imide diol 8 as an off white solid (108 g, 81%).

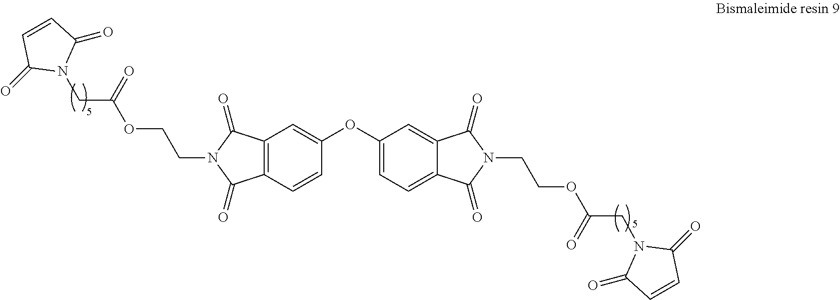

### Preparation of Inventive Bismaleimide Resin 9

In a 1 L 3 necked flask equipped with a mechanical stirrer and water condenser, were taken imide diol 8 (56.71 g, 143 mmol), 6-maleimidocaproic acid (72.6 g, 343 mmol), PTSA mono hydrate (2.71 g, 14.3 mmol), 4-methoxyphenol (120 mg, 1000 ppm) in toluene (400 mL). The mixture was refluxed with azeotrope distillation of water for about 8 h. After cooling to room tempertaure, the mixture was diluted with ethyl acetate, washed with aq. NaHCO₃ solution twice, deionized water until the ionic conductivity was about 2 uS. The organic layer was passed through a silica column containing a short plug of sillitin in between the silica layers. Another 500 ppm pf 4-methoxyphenol was added and the solvent evaporated on rotovap to give bismaleimide resin 9 as a brown viscous liquid (89 g, 79%), which solidified upon standing at room temperature.

1. A resin comprising the structure: 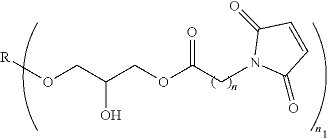
Wherein:
R is a multivalent hydrocarbyl linker selected from linear or branched alkyls, linear or branched cycloalkyls, alkylenes, cycloalkylenes, bicycloalkylenes, tricycloalkylenes, linear or branched alkylenes, linear or branched cycloalkylenes, linear or branched alkenylenes, arylenes, aralkylenes, arylbicycloalkylenes, aryltricycloalkylenes, bicycloalkylarylenes, tricycloalkylarylenes, bisphenylenes, cycloalkylarylenes, heterocycloalkylene or heterocycloarylenes; the alkyls, cycloalkyls, alkylenes, cycloalkylenes, alkenylenes, arylenes, aralkylenes, arylbicycloalkylenes, aryltricycloalkylenes, bicycloalkylarylenes, tricycloalkylarylenes, bisphenylenes, cycloalkylarylenes, heterocycloalkylene and heterocycloarylenes can optionally contain O or S or hydroxyl group; and
n and n₁ are each independently 1-10.

2. A resin comprising the structure: 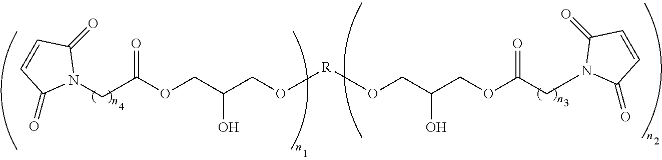
Wherein:
R is a multivalent hydrocarbyl linker selected from linear or branched alkyls, linear or branched cycloalkyls, alkylenes, cycloalkylenes, bicycloalkylenes, tricycloalkylenes, linear or branched alkylenes, linear or branched cycloalkylenes, linear or branched alkenylenes, arylenes, aralkylenes, arylbicycloalkylenes, aryltricycloalkylenes, bicycloalkylarylenes, tricycloalkylarylenes, bisphenylenes, cycloalkylarylenes, heterocycloalkylene or heterocycloarylenes; the alkyls, cycloalkyls, alkylenes, cycloalkylenes, alkenylenes, arylenes, aralkylenes, arylbicycloalkylenes, aryltricycloalkylenes, bicycloalkylarylenes, tricycloalkylarylenes, bisphenylenes, cycloalkylarylenes, heterocycloalkylene and heterocycloarylenes can optionally contain O or S or hydroxyl group; and
n₁, n₂, n₃, and n₄ are each independently 1-10.

3. A resin comprising the structure: 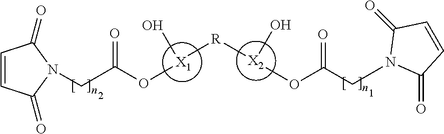
Wherein:
X₁ and X₂ are 3-10 membered ring groups independently selected from functionalized or unfunctionalized alicycyclic groups optionally having one or more heteroatoms;
n₁ and n₂ are each independently 1-10;
R is a multivalent hydrocarbyl linker selected from linear or branched alkyls, linear or branched cycloalkyls, alkylenes, cycloalkylenes, bicycloalkylenes, tricycloalkylenes, linear or branched alkylenes, linear or branched cycloalkylenes, linear or branched alkenylenes, arylenes, aralkylenes, arylbicycloalkylenes, aryltricycloalkylenes, bicycloalkylarylenes, tricycloalkylarylenes, bisphenylenes, cycloalkylarylenes, heterocycloalkylene or heterocycloarylenes; the alkyls, cycloalkyls, alkylenes, cycloalkylenes, alkenylenes, arylenes, aralkylenes, arylbicycloalkylenes, aryltricycloalkylenes, bicycloalkylarylenes, tricycloalkylarylenes, bisphenylenes, cycloalkylarylenes, heterocycloalkylene and heterocycloarylenes can optionally contain O or S or hydroxyl group; and
R is linked to the ring structures X₁ and X₂ at any position with a proviso that the hydroxyl groups on X₁ and X₂ rings are adjacent to the maleimidoalkanoyl groups

4. A resin comprising the structure: 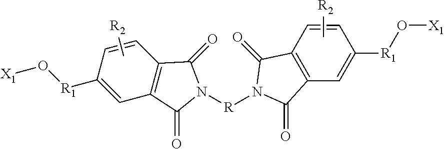
Wherein:
R is a multivalent hydrocarbyl linker selected from linear or branched alkyls, linear or branched cycloalkyls, alkylenes, cycloalkylenes, bicycloalkylenes, tricycloalkylenes, linear or branched alkylenes, linear or branched cycloalkylenes, linear or branched alkenylenes, arylenes, aralkylenes, arylbicycloalkylenes, aryltricycloalkylenes, bicycloalkylarylenes, tricycloalkylarylenes, bisphenylenes, cycloalkylarylenes, heterocycloalkylene or heterocycloarylenes; the alkyls, cycloalkyls, alkylenes, cycloalkylenes, alkenylenes, arylenes, aralkylenes, arylbicycloalkylenes, aryltricycloalkylenes, bicycloalkylarylenes, tricycloalkylarylenes, bisphenylenes, cycloalkylarylenes, heterocycloalkylene and heterocycloarylenes can optionally contain O or S or hydroxyl group;
R₁ is a carbonyl group; an aliphatic or aromatic linker which may contain one or more of ester, ether, hydroxyl or thioether groups;
R₂ is a substituent on the aromatic ring, which can be H, halogen, alkyl, alkyl ether, thioether group; and
X₁ is selected from maleimidoalkanoyl or maleimidoaroyl group.

5. A resin comprising the structure 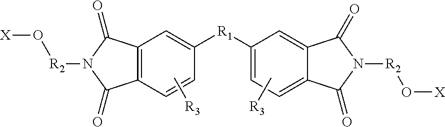
Wherein:
R₁ can be just a bond linking the two aromatic groups; O; carbonyl; or a multivalent hydrocarbyl linker selected from linear or branched alkyls, linear or branched cycloalkyls, alkylenes, cycloalkylenes, bicycloalkylenes, tricycloalkylenes, linear or branched alkylenes, linear or branched cycloalkylenes, linear or branched alkenylenes, arylenes, aralkylenes, arylbicycloalkylenes, aryltricycloalkylenes, bicycloalkylarylenes, tricycloalkylarylenes, bisphenylenes, cycloalkylarylenes, heterocycloalkylene or heterocycloarylenes; the alkyls, cycloalkyls, alkylenes, cycloalkylenes, alkenylenes, arylenes, aralkylenes, arylbicycloalkylenes, aryltricycloalkylenes, bicycloalkylarylenes, tricycloalkylarylenes, bisphenylenes, cycloalkylarylenes, heterocycloalkylene and heterocycloarylenes can optionally contain O or S or hydroxyl group;
R₂ is an aliphatic or aromatic linker group which may contain one or more of ester, ether, hydroxyl, thioether or carbonate groups;
R₃ is a substituent on the aryl group, which may be H, halogen, alkyl, alkyl ether, or thio ether group; and
X is a polymerizable functionality selected from maleimidoalkanoyl and maleimidoaroyl groups.

6. A resin comprising the structure 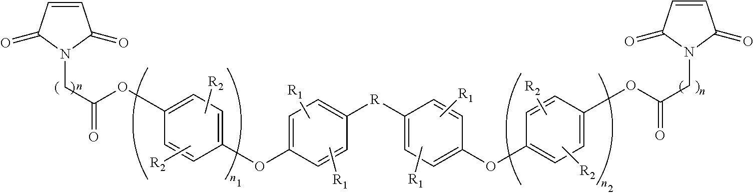
Wherein:
R is a divalent hydrocarbyl linker selected from linear or branched alkyls, linear or branched cycloalkyls, alkylenes, cycloalkylenes, bicycloalkylenes, tricycloalkylenes, linear or branched alkylenes, linear or branched cycloalkylenes, linear or branched alkenylenes, arylenes, aralkylenes, arylbicycloalkylenes, aryltricycloalkylenes, bicycloalkylarylenes, tricycloalkylarylenes, bisphenylenes, cycloalkylarylenes, heterocycloalkylene or heterocycloarylenes; the alkyls, cycloalkyls, alkylenes, cycloalkylenes, alkenylenes, arylenes, aralkylenes, arylbicycloalkylenes, aryltricycloalkylenes, bicycloalkylarylenes, tricycloalkylarylenes, bisphenylenes, cycloalkylarylenes, heterocycloalkylene and heterocycloarylenes can optionally contain O or S or hydroxyl group;
R₁ and R₂ are each linear or branched aliphatic groups optionally containing heteroatoms;
n=1-10; and
n₁ and n₂ are 1-100.
